(19) United States
(12) Patent Application Publication
(10) Pub. No.: US 2013/0072797 A1
(43) Pub. Date: Mar. 21, 2013

(54) 3D ULTRASOUND APPARATUS AND METHOD FOR OPERATING THE SAME

(71) Applicant: SAMSUNG MEDISON CO., LTD., Gangwon-do (KR)

(72) Inventor: Kwang-hee LEE, Gangwon-do (KR)

(73) Assignee: SAMSUNG MEDISON CO., LTD., Gangwon-do (KR)

(21) Appl. No.: 13/669,097

(22) Filed: Nov. 5, 2012

Related U.S. Application Data
(63) Continuation-in-part of application No. 13/010,310, filed on Jan. 20, 2011.

(30) Foreign Application Priority Data
May 31, 2010 (KR) 10-2010-0051144

(51) Int. Cl. A61B 8/00; A61B 8/08; A61B 8/13

(52) U.S. Cl. CPC A61B 8/466; A61B 8/13; A61B 8/5207; A61B 8/54; A61B 8/5292
USPC 600/443

(57) ABSTRACT

A 3-dimensional (3D) ultrasound apparatus and a method for operating the same are provided. A 3D ultrasound apparatus includes a first processor, a second processor and a controller. The first processor determines a start point from an image data obtained by scanning an object in a human body. The second processor extracts a top image of the object from the image data based on the start point. The controller controls a sagittal view of the object by rotating the image data, using the top image.

### CROSS-REFERENCE TO RELATED APPLICATION

This application claims the benefit of Korean Patent Application No. 10-2010-0051144, filed on May 31, 2010, in the Korean Intellectual Property Office, the disclosure of which is incorporated herein by reference.

### BACKGROUND

1. Field of the Invention

The present invention relates to a 3-dimensional (3D) ultrasound apparatus and a method for operating the same, in which a top image of an object in a human body is extracted from an image data based on a start point in the image data obtained by scanning the object, and the image data is rotated using the extracted top image, thereby automatically determining a sagittal view of the object.

2. Description of the Related Art

An ultrasound system is an apparatus that irradiates an ultrasound signal from a surface of a human body towards a target part, that is, an object such as a fetus, an internal organ, and the like, under the body surface and obtains an image of a monolayer or blood flow in soft tissue from information in the reflected ultrasound signal. The ultrasound system has been widely used together with other image diagnostic systems such as X-ray diagnostic systems, computerized tomography (CT) scanners, magnetic resonance image (MRI) systems and nuclear medicine diagnostic systems because of its various merits such as a small size, a low price, real-time image display, and high stability through elimination of any radiation exposure.

Also, a general method for diagnosing a Down's syndrome fetus is to measure the thickness of a fetus' nuchal translucency (NT) through the ultrasound system. The method was developed by Nicolaides in 1992, and it has been known that in a case where a fetus has an abnormal symptom, body fluid is accumulated in subcutaneous tissues at the nape of a fetus' neck and therefore, the fetus' NT of the fetus becomes thick.

Another method for diagnosing a Down's syndrome fetus is to measure the angle between a fetus' maxilla and nasal bone, that is, the frontmaxillary facial (FMF) angle, and the like. For example, in a case where the FMF angle of a fetus is greater than 88.7 degrees as compared with 78.1 that is the FMF angle of a normal fetus, it is highly likely that the fetus has Down's syndrome.

Therefore, in order to easily diagnose a Down's syndrome fetus, it is required to more easily and precisely inspect the thickness of the fetus' NT or the angle between the fetus' maxilla and nasal bone.

However, since a measured value changes depending on the position or angle of a fetus, the sagittal view for the fetus should be precisely controlled so as to properly inspect the thickness of the fetus' NT or the angle between the fetus' maxilla and the fetus' nasal bone.

Accordingly, it is required to develop a technology for precisely controlling the sagittal view for a fetus.

### SUMMARY

An aspect of the present invention provides a 3-dimensional (3D) ultrasound apparatus and a method for operating the same, in which a top image of an object in a human body is extracted from an image data based on a start point in the image data obtained by scanning the object, and the image data is rotated using the extracted top image, thereby automatically determining a sagittal view of the object.

An aspect of the present invention also provides a 3D ultrasound apparatus and a method for operating the same, in which, in a case where an object in a human body is a fetus, a top image of the object corresponding to a basic data on the rotation of an image data is easily extracted from the image data obtained by scanning the object, using the direction of the fetus' head, thereby rapidly controlling a sagittal view of the object.

An aspect of the present invention also provides a 3D ultrasound apparatus and a method for operating the same, in which a mid sagittal plane with respect to an object is effectively detected by detecting symmetry window regions parallel to each other by using a start point and calculating similarity between the symmetry window regions.

According to an aspect of the present invention, there is provided a 3D ultrasound apparatus, the apparatus including a first processor to determine a start point from an image data obtained by scanning an object in a human body, a second processor to extract a top image of the object from the image data based on the start point, and a controller to control a sagittal view of the object by rotating the image data, using the top image.

According to another aspect of the present invention, there is provided a method for operating a 3D ultrasound apparatus, the method including determining a start point from an image data obtained by scanning an object in a human body, extracting a top image of the object from the image data based on the start point, and rotating the image data using the top image, thereby controlling a sagittal view of the object.

According to another aspect of the present invention, there is provided a method for operating a 3D ultrasound apparatus, the method including: determining a start point from image data obtained by scanning an object in a human body; and detecting a mid sagittal plane with respect to the object from the image data by using similarity between two symmetry window regions parallel to a reference side image where the start point is located.

According to another aspect of the present invention, there is provided a 3D ultrasound apparatus, the apparatus including: a first processor to determine a start point from image data obtained by scanning an object in a human body; and a controller to detect a mid sagittal plane with respect to the object from the image data by using similarity between two symmetry window regions parallel to a reference side image where the start point is located.

According to another aspect of the present invention, there is provided a method for operating a 3D ultrasound apparatus, the method including: determining a start point from image data obtained by scanning an object in a human body; extracting a top image with respect to the object from the image data, based on the start point; matching a template with respect to the object indicated on an initial top image where the start point is located; and detecting a mid sagittal plane with respect to the object from the image data, by using similarity between symmetry window regions obtained based on the template and a reference side image where the start point is located.

According to another aspect of the present invention, there is provided a 3D ultrasound apparatus, the apparatus including: a first processor to determine a start point from image data obtained by scanning an object in a human body; a second processor to extract a top image with respect to the object from the image data, based on the start point, and match a template with respect to the object indicated on an initial top image where the start point is located; and a controller to detect a mid sagittal plane with respect to the object from the image data, by using similarity between symmetry window regions obtained based on the template and a reference side image where the start point is located.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a block diagram illustrating a configuration of a 3-dimensional (3D) ultrasound apparatus according to an embodiment of the present invention;

FIG. 2 is a diagram illustrating an example of an image for each direction with respect to an object extracted in a 3D ultrasound apparatus according to the embodiment of the present invention;

FIG. 3 is a diagram illustrating a method for determining a start point from an image data obtained by scanning an object, using a 3D ultrasound apparatus according to the embodiment of the present invention;

FIG. 4 is a diagram illustrating a method for determining the direction of an object, using a 3D ultrasound apparatus according to the embodiment of the present invention;

FIG. 5 is a diagram illustrating an example of extracting a top image for an object as a basic data for controlling a sagittal view, using a 3D ultrasound apparatus according to the embodiment of the present invention;

FIG. 6 is a diagram illustrating an example of correcting a front image for an object, using a 3D ultrasound apparatus according to the embodiment of the present invention;

FIG. 7 is a diagram illustrating an example of controlling a sagittal view by rotating an image data for an object, using a 3D ultrasound apparatus according to the embodiment of the present invention;

FIG. 8 is a flowchart illustrating a method for operating a 3D ultrasound apparatus according to an embodiment of the present invention;

FIG. 9 is a block diagram illustrating a configuration of a 3D ultrasound apparatus according to another embodiment of the present invention;

FIG. 10 is a flowchart illustrating a method for operating a 3D ultrasound apparatus according to another embodiment of the present invention; and

FIGS. 11A and 11B are diagrams illustrating an example of detecting a mid sagittal plane according to the embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

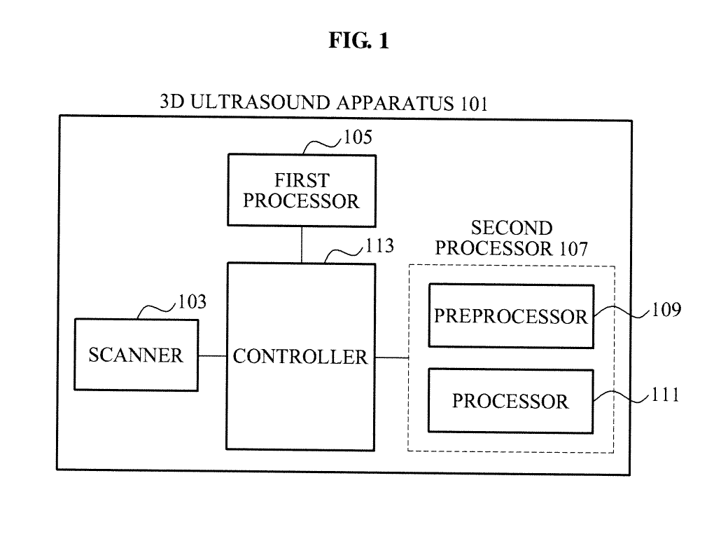

FIG. 1 is a block diagram illustrating a configuration of a 3-dimensional (3D) ultrasound apparatus 101 according to an embodiment of the present invention.

Referring to FIG. 1, the 3D ultrasound apparatus 101 includes a scanner 103, a first processor 105, a second processor 107, and a controller 113.

The scanner 103 may extract at least one of a side image (A-plane in a side direction or a sagittal plane), a top image (B-plane in a top direction or a transverse plane) and a front image (C-plane in a front direction or a coronal plane) with respect to an object in a human body from an image data obtained by scanning the object and then may display the at least one of the side image, the top image, and the front image on a screen. In this instance, the scanner 103 may remove noise from each of the side image, the top image, and the front image so that the contours of images with respect to the object are clearly displayed on the screen.

The first processor 105 determines a start point from the image data obtained by scanning an object in a human body. Here, the object in the human body may include a fetus, an internal organ, and the like. In a case where the object is a fetus, the first processor 105 may extract a side image of the fetus from the image data and identify the fetus' nasal bone. Then, the first processor 105 may determine a start point using the fetus' nasal bone.

Specifically, the first processor 105 may extract a side image of the fetus from the image data and identify the fetus' nasal bone or maxilla by using the intensity of the side image. In this instance, the first processor 105 may place a seed at the fetus' nuchal translucency (NT) and set a window area based on the seed. Then, the first processor 105 may identify a part in the window area of which the intensity is highest as the fetus' nasal bone or maxilla while moving the window area upward. The intensity being the highest is a result of bone being reflected most strongly and thus, an area in which the bone is placed appears most bright.

The first processor 105 may identify the frontmaxillary facial (FMF) angle between the fetus' nasal bone and maxilla, using the fetus' nasal bone and maxilla.

Subsequently, the first processor 105 may determine a virtual point as the start point. Here, the virtual point is spaced apart upward by a selected distance, for example, 1.3 cm to 1.5 cm, from a point at which a vertical line that passes through an end point of the fetus' nasal bone is intersected with a horizontal line that passes through the fetus' NT.

Alternatively, the first processor 105 may determine the start point based on a user input externally received. In other words, the first processor 105 may determine the start point even via the user input selecting any one location of the image data through any one of various types of devices, such as a keypad, a mouse, a track ball, and a touch screen.

The second processor 107 extracts a top image of the object from the image data based on the start point. Specifically, the second processor includes a preprocessor 109 and a processor 111.

In a case where the object is a fetus, the preprocessor 109 may determine the direction of the fetus' head. For example, the preprocessor 109 moves a first virtual plane in a side direction at a predetermined interval in a direction perpendicular to the first virtual plane in the side direction with respect to the image data, thereby extracting a plurality of image data included in the first virtual plane. Subsequently, the preprocessor 109 may identify the direction of the FMF angle between the fetus' nasal bone and maxilla from the plurality of image data included in the first virtual plane. In a case where an amount of image data including an FMF angle in a first direction, for example, a left direction, is greater than an amount of image data including an FMF angle in a second direction, for example, a right direction, the preprocessor 109 may determine the first direction as the direction of the fetus' head.

The processor 111 moves a second virtual plane in a top direction at a predetermined interval in the direction of the fetus' head from the start point with respect to the image data, thereby extracting a plurality of image data included in the second virtual plane.

Subsequently, the processor 111 may extract any one of the plurality of image data included in the second virtual plane as the top image. In this instance, the processor 111 may measure the outer circumferences of images from the image data included in the second virtual plane and may select each image data having a larger circumference than the mean of the measured outer circumferences for all image data. Then, the processor 111 may extract, as the top image, an image data having the smallest template matching among each of the image data having a larger circumference than the mean of the measured outer circumferences.

For example, in a case where the object is a fetus, the processor 111 may measure the circumferences of ellipses corresponding to the fetus' head from the plurality of image data included in the second virtual plane, and extract, as the top image, an image data having the smallest template matching among image data each having a larger circumference than the mean of the measured circumferences of the ellipses. In this instance, the processor 111 may extract an image data having the circumference of an ellipse, relatively highly matched to a selected template, for example, an ellipse having an occipitofrontal diameter (OFD) of 2.5 cm and an aspect ratio of 1.5, as the top image.

Accordingly, the processor 111 moves the second virtual plane in the direction of the fetus' head from the start point, so as to more rapidly extract the top image as compared with a case that the second virtual plane is moved with respect to the entire fetus from the fetus' tiptoe to head.

The controller 113 may identify the fetus' nasal bone by using the intensity of the side image of the fetus, extracted from the image data, and may move one side of the image data in front and vertical directions so that the fetus' nasal bone is placed at the highest position. In this instance, the controller 113 controls the image of the fetus not to be diagonally placed by moving the one side of the image data in the front and vertical directions so that the fetus' nasal bone is placed at the highest position. Accordingly, the image of the fetus can be placed bilaterally symmetric in the front image of the fetus.

The controller 113 may control a sagittal view of the object by rotating the image data using the top image. In this instance, the controller 113 may pass through an arbitrary point in the second virtual plane in the top direction and rotate the image data with respect to a virtual axis that passes through the side image.

Thus, the controller 113 rotates the image data, using the intensity of an image included in the side image or the left/right matching of the appearance of an image included in the top image, thereby automatically controlling the sagittal view of the object.

1) Rotation of Image Data Using Falx Area

In a case where the object is a fetus, the controller 113 may extract a side image of the fetus from the image data and rotate the image data so that the brightness intensity in a falx area of the fetus, included in the side image, is largest.

Here, where the side image is a mid-sagittal, a part of the fetus, that is, the falx area is uniformly distributed bright. Conversely, where the side image is not a mid-sagittal, the falx area is not uniformly bright, and a dark area appears.

Accordingly, using a characteristic of brightness as described above, the controller 113 may rotate the image data so that the falx area is most brightly and uniformly distributed while moving and rotating an ultrasound data with respect to the center of the fetus' head.

2) Rotation of Image Data Using Left/Right Matching Degree

The controller 113 may automatically control a sagittal view of the object by matching a figure corresponding to the fetus included in the top image and rotating the image data so that the left/right matching of the matched figure is highest.

For example, in a case where the matched figure is an ellipse, the controller 113 may vertically place the major axis of the ellipse, and rotate the image data so that the left and right of the ellipse are most symmetric with respect to the major axis.

According to the present embodiment, a top image of an object is extracted from an image data based on a start point of the image data obtained by scanning the object in a human body, and the image data is rotated using the extracted top image, so that a sagittal view of the object can be automatically determined.

Also, in a case where the object in the human body is a fetus, a top image of the object corresponding to a basic data on the rotation of the image data from the image data obtained by scanning the object, using the direction of the fetus' head, so that the sagittal view of the object can be rapidly controlled.

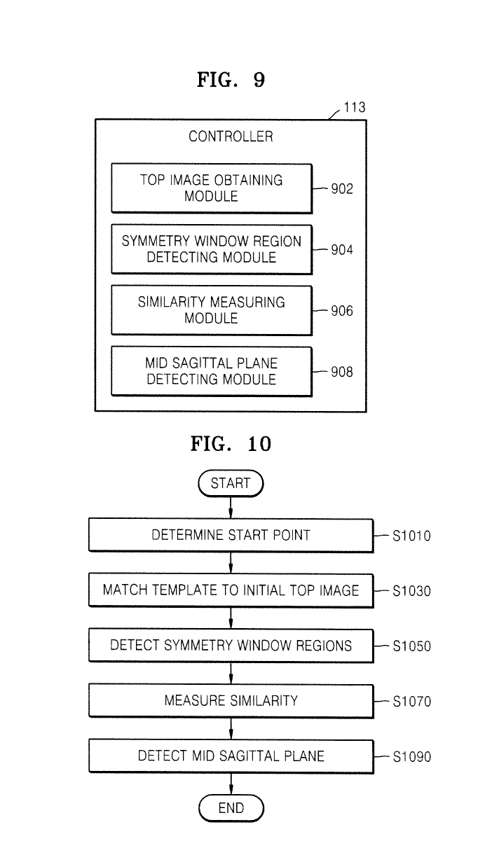

Meanwhile, the controller 113 may match a figure or a template to the top image, and detect two symmetry window regions parallel to each other by using the matched figure or template. Then, the controller 113 may measure similarity between the symmetry window regions and detect a reference side image when the similarity is highest as a mid sagittal plane. An example of detecting a mid sagittal plane will be described in detail later with reference to FIGS. 9 through 11B.

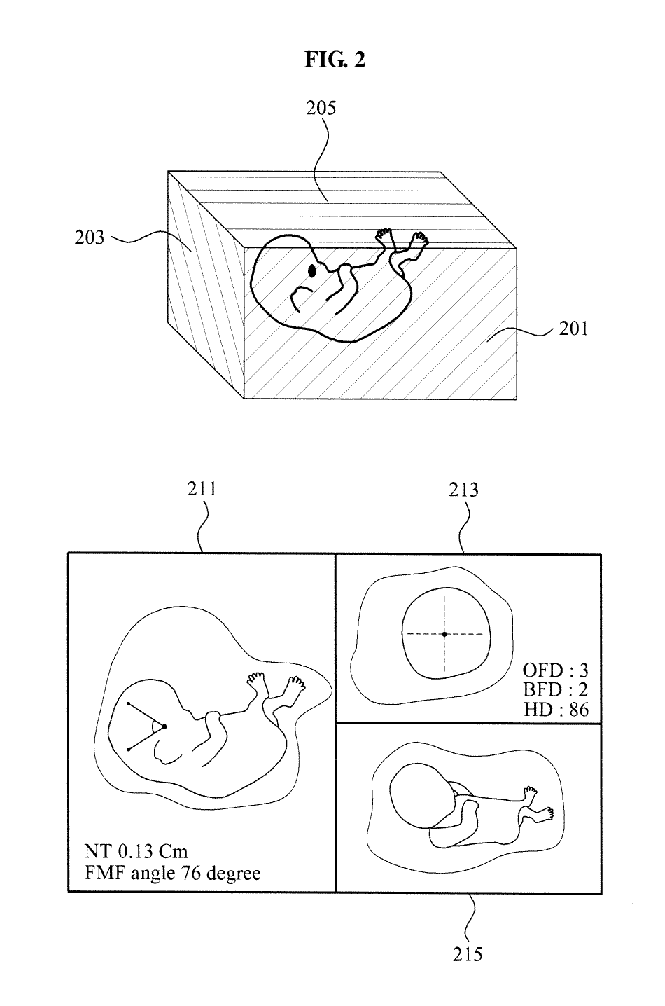

FIG. 2 is a diagram illustrating an example of an image for each direction with respect to an object extracted in a 3D ultrasound apparatus according to the embodiment of the present invention.

Referring to FIG. 2, the 3D ultrasound apparatus may extract a side image, a top image, or a front image from an image data obtained by scanning an object in a human body and may display the image on the screen.

For example, the 3D ultrasound apparatus may extract a side image in a side direction, which displays a ‘first plane’ 201, from an image data obtained by scanning an object and display the side image in a first area 211 on the screen. The 3D ultrasound apparatus may extract a top image in a top direction, which displays a ‘second plane’ 203, from the image data obtained by scanning the object, and may display the top image in a second area 213 on the screen. The 3D ultrasound apparatus may extract a front image in a front direction, which displays a ‘third plane’ 205, from the image data obtained by scanning the object, and may display the front image in a third area 215 on the screen.

As the image data is rotated or moved based on a selected reference, the 3D ultrasound apparatus updates the side image, the top image, or the front image and displays the updated image on the screen, thereby easily detecting a 3D object.

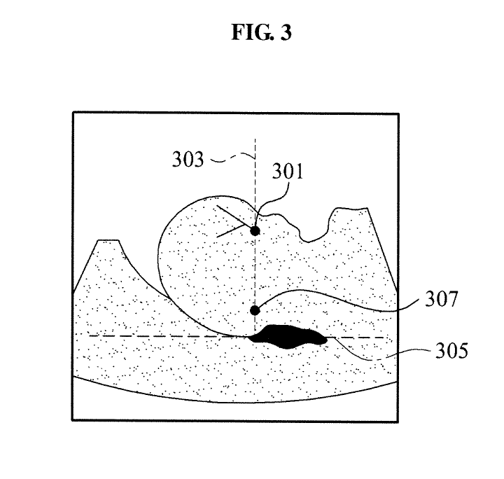

FIG. 3 is a diagram illustrating a method for determining a start point from an image data obtained by scanning an object, using a 3D ultrasound apparatus according to the embodiment of the present invention.

Referring to FIG. 3, the 3D ultrasound apparatus may extract a side image of a fetus from an image data obtained by scanning the fetus, and may identify the fetus' nasal bone or maxilla by using the intensity of the side image. For example, the 3D ultrasound apparatus may identify a part of the side image of which intensity is highest as the fetus' nasal bone or maxilla.

In this instance, the 3D ultrasound apparatus may determine a virtual point 307 as a start point. Here, the virtual point 307 is spaced apart upward by a selected distance from a point at which a vertical line 303 that passes through an end point 301 of the fetus' nasal bone is intersected with a horizontal line 305 that passes through fetus' NT.

Alternatively, as described above with reference to FIG. 1, the 3D ultrasound apparatus may determine the start point according to a user input.

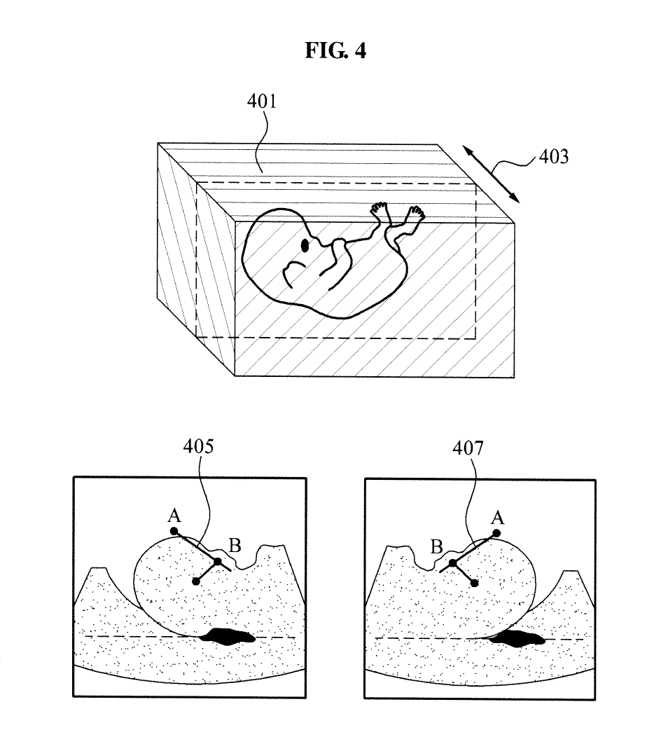

FIG. 4 is a diagram illustrating a method for determining the direction of an object, using a 3D ultrasound apparatus according to the embodiment of the present invention.

Referring to FIG. 4, in a case where the object is a fetus, the 3D ultrasound apparatus may determine the direction of fetus' head from an image data by scanning the fetus.

For example, the 3D ultrasound apparatus moves a first virtual plane 401 in a side direction in a direction 403 perpendicular to the first virtual plane 410 at a predetermined interval with respect to the image data, thereby extracting a plurality of image data included in the first virtual plane 401. In this instance, the 3D ultrasound apparatus may apply a top-hat transform to the image data so as to precisely and easily extract the fetus' nasal bone and maxilla.

Subsequently, the 3D ultrasound apparatus identifies the direction of the FMF angle between the fetus' nasal bone and maxilla from the plurality of image data included in the first virtual plane 401.

In a case where an amount of image data including an FMF angle 405 in a first direction, for example, a left direction, are greater than an amount of image data including an FMF angle 407 in a second direction, for example, a right direction, the 3D ultrasound apparatus may determine the first direction as the direction of the fetus' head. Specifically, the 3D ultrasound apparatus may provide grades as ‘left:right=7:3’ with respect to the direction of the fetus' head, estimated based on the plurality of image data. Finally, the 3D ultrasound apparatus may determine the direction of the fetus' head as the left direction to which a relatively high grade is provided.

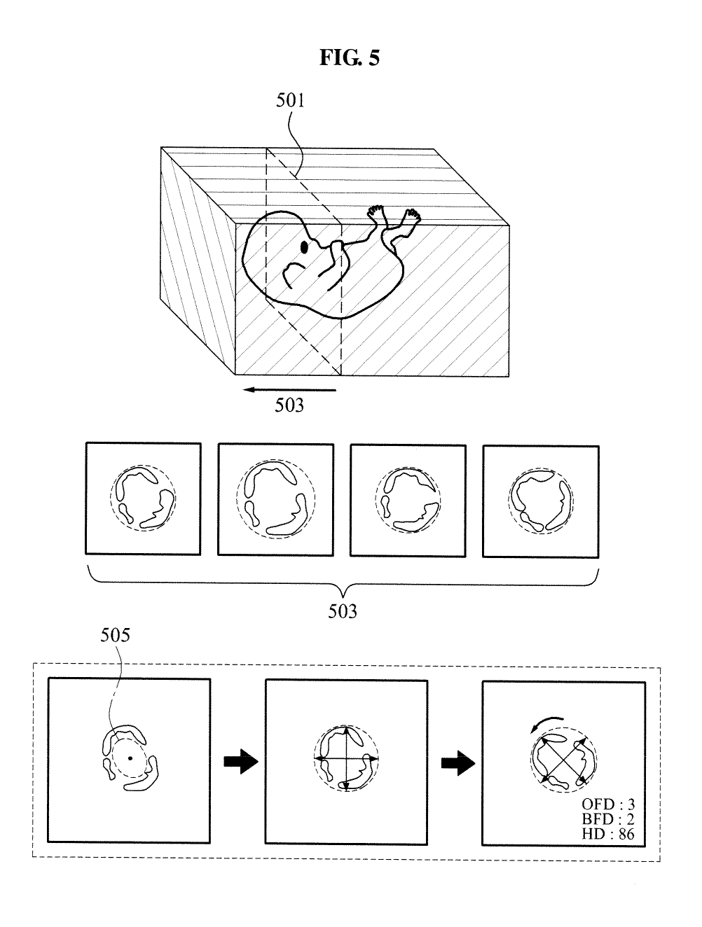

FIG. 5 is a diagram illustrating an example of extracting a top image for an object as a basic data for controlling a sagittal view, using the 3D ultrasound apparatus according to the embodiment of the present invention.

Referring to FIG. 5, the 3D ultrasound apparatus moves a second virtual plane 501 in a top direction in the direction 503 of fetus' head at a predetermined interval from a start point 501 with an image data obtained by scanning a fetus, thereby extracting a plurality of image data included in the second virtual plane 501.

Subsequently, the 3D ultrasound apparatus measures the circumferences of ellipses corresponding to the fetus' head from the image data included in the second virtual plane 501, and determines an image data having a larger circumference than the mean of the measured circumferences of the ellipses. For example, in a case where the number of image data included in the second virtual plane 501 is 10, the 3D ultrasound apparatus may determine four image data each having a larger circumference than the mean of the circumferences of ellipses, that is, 8.6 cm.

The 3D ultrasound apparatus may extract, as a top image, an image data having the smallest template matching among the image data each having a larger circumference than the mean of the circumferences of the ellipses. For example, the 3D ultrasonic apparatus may extract, as the top image, one image data having a circumference highly matched to a selected template, for example, an ellipse having an OFD of 2.5 cm and an aspect ratio of 1.5, among the four image data each having a larger circumference than the mean of the circumferences of the ellipses, that is, 8.6 cm.

Here, the 3D ultrasound apparatus may display an ellipse template 505 on an ellipse corresponding to the fetus' head in each of the image data, and may change the biparietal diameter (BPD) or OFD of the ellipse template 505 so that the ellipse template 505 is matched to the ellipse corresponding to the fetus' head. In this instance, the 3D ultrasound apparatus may extract an image data most highly matched to the ellipse template 505 by minimizing the change of the ellipse template 505.

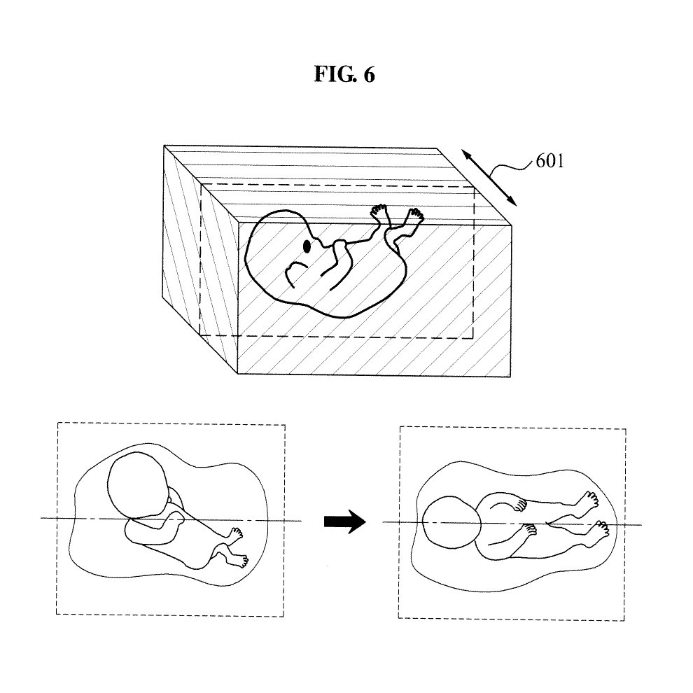

FIG. 6 is a diagram illustrating an example of correcting a front image for an object, using a 3D ultrasound apparatus according to the embodiment of the present invention.

Referring to FIG. 6, the 3D ultrasound apparatus may extract a side image of a fetus from an image data obtained by scanning the fetus, and move one side of the image data in a direction 601 perpendicular to the side image so that the fetus' nasal bone is placed at the highest position in the side image.

In this instance, the 3D ultrasound apparatus controls the image of the fetus not to be diagonally placed by moving the one side of the image data in the direction 601 perpendicular to the side image so that the fetus' nasal bone is placed at the highest position. Accordingly, the 3D ultrasound apparatus may display a front image so that the fetus is placed bilaterally symmetric in the front image of the fetus. That is, the 3D ultrasound apparatus may display the front image so that the fetus' face, arms, and legs are placed bilaterally symmetric in the front image.

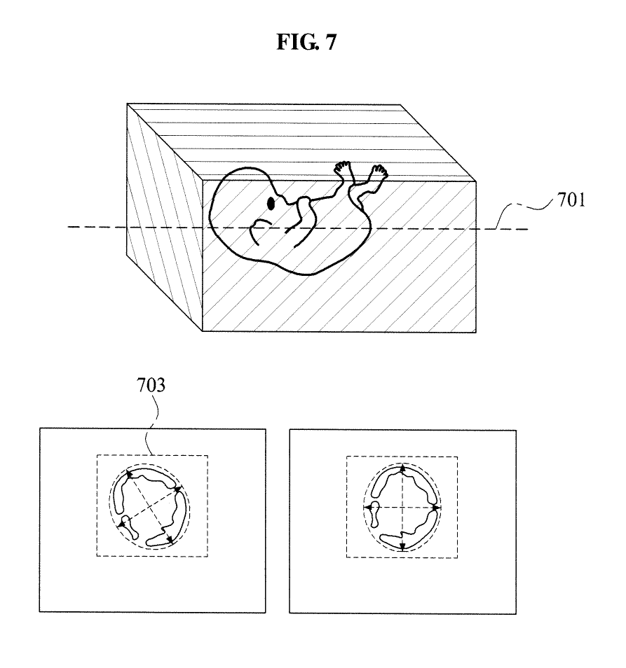

FIG. 7 is a diagram illustrating an example of controlling a sagittal view by rotating an image data for an object, using a 3D ultrasound apparatus according to the embodiment of the present invention.

Referring to FIG. 7, the 3D ultrasound apparatus extracts a top image of an object from an image data obtained by scanning the object and rotates the image data using the top image, thereby controlling a sagittal view of the object.

For example, the 3D ultrasound apparatus may automatically control a sagittal view of the object by setting a window area 703 in the top image, and by matching a figure corresponding to the object included in the top image and rotating the image data so that the left/right matching of the matched figure is highest. That is, in a case where the figure is an ellipse, the 3D ultrasound apparatus may vertically place the major axis of the ellipse and rotate the image data so that the left and right of the ellipse are most symmetric with respect to the major axis.

In a case where the ellipse of the top image is inclined, the 3D ultrasound apparatus controls the ellipse of the top image not to be inclined by rotating the image data with respect to a virtual axis 701 that passes through an arbitrary point in the second virtual plane in the top direction and passes the side image. Accordingly, the left/right matching of the circumference of the ellipse may be increased.

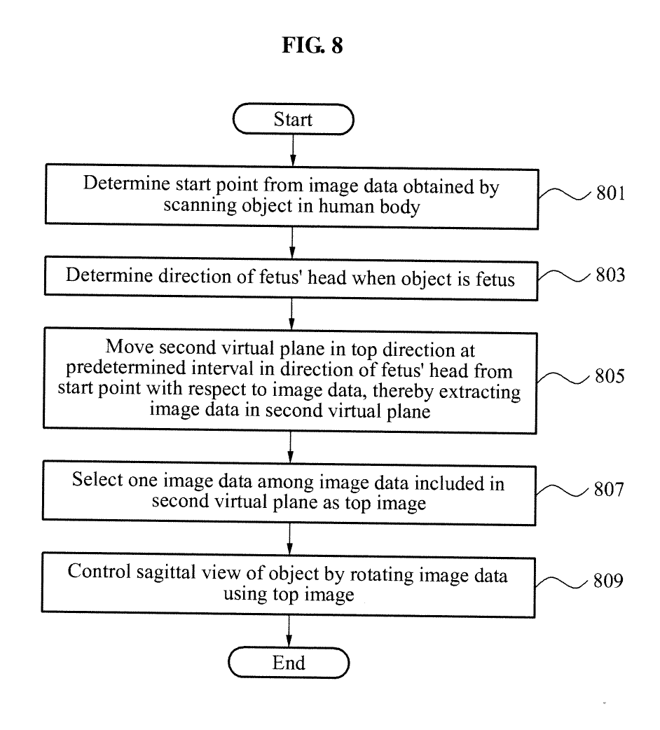

FIG. 8 is a flowchart illustrating a method for operating a 3D ultrasound apparatus according to an embodiment of the present invention.

Referring to FIG. 8, in operation 801, the 3D ultrasound apparatus determines a start point from an image data obtained by scanning an object in a human body.

In a case where the object is a fetus, the 3D ultrasound apparatus may extract a side image of the fetus from the image data and identify the fetus' nasal bone. Then, the 3D ultrasound apparatus may determine a start point using the fetus' nasal bone.

First, the 3D ultrasound apparatus may extract a side image of the fetus from the image data and identify fetus' nasal bone or maxilla by using the intensity of the side image. As bone is reflected most strongly, an area in which the bone is placed appears most bright. For this reason, the 3D ultrasound apparatus may identify a part of the side image of which intensity is highest as the fetus' nasal bone or maxilla.

Subsequently, the 3D ultrasound apparatus may determine a virtual point as the start point. Here, the virtual point is spaced apart upward by a selected distance, for example, 1.3 cm to 1.5 cm, from a point at which a vertical line that passes through an end point of the fetus' nasal bone is intersected with a horizontal line that passes through the fetus' NT. In operation 803, in a case where the object is a fetus, the 3D ultrasound apparatus determines the direction of fetus' head.

For example, the 3D ultrasound apparatus moves a first virtual plane in a side direction at a predetermined interval in a direction perpendicular to the first virtual plane with respect to the image data, thereby extracting a plurality of image data included in the first virtual plane.

Subsequently, the 3D ultrasound apparatus may identify the direction of the FMF angle between the fetus' nasal bone and maxilla from the plurality of image data included in the first virtual plane. In a case where an amount of image data including an FMF angle in a first direction, for example, a left direction, are greater than an amount of image data including an FMF angle in a second direction, for example, a right direction, the 3D ultrasound apparatus may determine the first direction as the direction of the fetus' head.

In operation 805, the 3D ultrasound apparatus moves a second virtual plane in a top direction at a predetermined interval in the direction of the fetus' head from the start point with respect to the image data, thereby extracting a plurality of image data included in the second virtual plane.

In operation 807, the 3D ultrasound apparatus selects one image data among the image data included in the second virtual plane as the top image.

In this instance, the 3D ultrasound apparatus measures outer circumferences of an image from the image data included in the second virtual plane and calculates the mean of the measured outer circumferences. The 3D ultrasound apparatus may select each image data having a larger circumference than the mean of the measured outer circumferences for all image data and extract, as the top image, an image data having the smallest template matching among the image data each having a larger circumference than the mean of the outer circumferences.

In a case where the fetus is diagonally placed, the 3D ultrasound apparatus may move the image data using the fetus' nasal bone in the side image of the fetus. That is, the 3D ultrasound apparatus may control the fetus to be placed bilaterally symmetric in the front image of the fetus by moving one side of the image data in a direction perpendicular to the side image so that the fetus' nasal bone is placed at the highest position.

In operation 809, the 3D ultrasound apparatus controls a sagittal view of the object by rotating the image data using the top image. In this instance, the 3D ultrasound apparatus may pass through an arbitrary point in the second virtual plane in the top direction and rotate the image data with respect to a virtual axis that passes through the side image.

Specifically, in a case where the object is a fetus, the 3D ultrasound apparatus may control a sagittal view of the object by extracting a side image of the fetus from the image data and by rotating the image data so that the brightness intensity in a falx area of the fetus, included in the side image, is largest.

Alternatively, in a case where the object is a fetus, the 3D ultrasound apparatus may control a sagittal view of the object by matching a figure corresponding to the fetus included in a top image and rotating the image data so that the left/right matching of the matched figure is highest.

FIG. 9 is a block diagram illustrating a configuration of a 3D ultrasound apparatus according to another embodiment of the present invention. In FIG. 9, the configuration of the controller 113 of the 3D ultrasound apparatus 101 of FIG. 1 is shown in detail.

According to the present embodiment, the controller 113 for detecting a mid sagittal plane may include a top image obtaining module 902, a symmetry window region detecting module 904, a similarity measuring module 906, and a mid sagittal plane detecting module 908. Hereinafter, an example of the controller 113 detecting a mid sagittal plane through each module will be described.

First, as described above with reference to FIGS. 1 through 8, the 3D ultrasound apparatus determines the start point. In other words, the first processor 105 of the 3D ultrasound apparatus may determine the virtual point 307 in the side image as the start point as described with reference to FIG. 3, or may determine the start point according to the user input.

The start point is a basis for the 3D ultrasound apparatus to perform rotation transformation or translation transformation on the image data, and may be determined to be at any location in the object of the image data. For example, the start point may be within a thalamus region.

The top image obtaining module 902 obtains the top image used to detect the mid sagittal plane. The top image obtaining module 902 may obtain the top image detected by the processor 111 of the second processor 107 described above with reference to FIGS. 1 through 8, or may obtain a new top image.

When the top image is obtained from the processor 111, the top image obtaining module 902 obtains the top image selected by the processor 111 based on the description above. Alternatively, the top image obtaining module 902 may obtain the top image including the start point determined by the first processor 105. In other words, the top image obtaining module 902 may obtain the top image including the start point as a transverse plane for determining the mid sagittal plane. Hereinafter, the top image including the start point for determining the mid sagittal plane may be referred to as an initial transverse plane.

The symmetry window region detecting module 904 detects the symmetry window regions parallel to each other by using the start point and the initial transverse plane. In detail, the symmetry window region detecting module 904 obtains a figure or a template matching the initial transverse plane. If the top image obtaining module 902 obtained the top image detected by the processor 111, the symmetry window region detecting module 904 may use information about a template used in the processor 111.

Then, the symmetry window region detecting module 904 may obtain the side image including the start point, and detect the two symmetry window regions parallel to the side image by using the information about the template matched to the initial transverse plane. Hereinafter, the side image including the start point will be referred to as an initial sagittal plane.

The symmetry window region detecting module 904 may detect two sagittal planes located at the same distance from the initial sagittal plane and parallel to each other (i.e., parallel to the initial sagittal plane), wherein a region included in the two sagittal planes is a symmetry window region. The initial sagittal plane changes as the image data rotates or moves, and a sagittal plane located at the same distance from the two symmetry window regions is referred to as a reference sagittal plane.

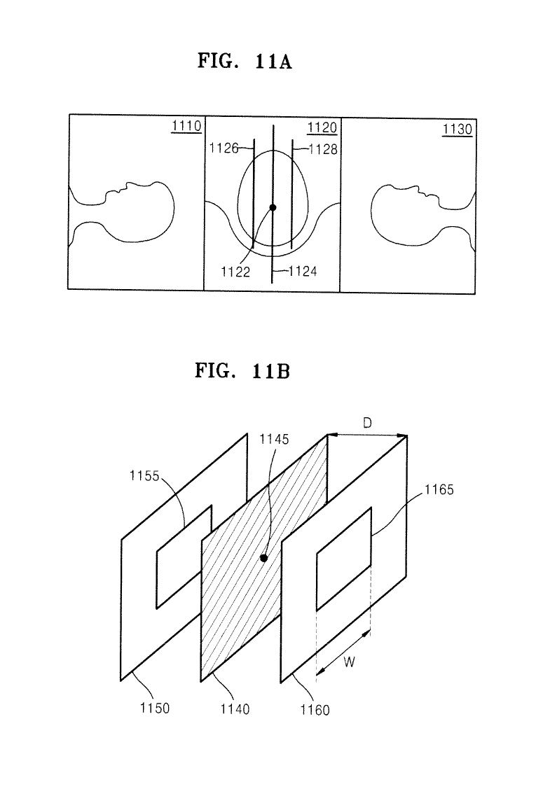

Meanwhile, a distance D from the reference sagittal plane to the symmetry window region and a length W of the symmetry window region may be determined according to a size or length of the template matched to the top image. For example, the symmetry window region detecting module 904 may detect the symmetry window region by using a size or length in a major axis of the template when the template is oval. A relationship between information about the template and the symmetry window region will be described in detail later with reference to FIG. 11.

The similarity measuring module 907 measures similarity between the symmetry window regions. In other words, the similarity measuring module 906 may measure a similarity value indicating a degree of similarity of the symmetry window regions by using a similarity function for comparing information about pixels included in the symmetry window regions. An example of the similarity function includes a normalized cross correlation (NCC) function, but the similarity measuring module 906 may use any type of similarity function other than the NCC function.

Meanwhile, in order to improve reliability of the similarity function, the similarity measuring module 906 may apply a support weight map about pixels in the symmetry window regions, along with the similarity function. The support weight map is a probability function about at least one of similarity consistency, distinctiveness, and proximity. In order to reduce effects of outlier and noise that are included in the symmetry window region but are not to be measured, the similarity measuring module 906 may use the similarity function and the support weight map together.

In addition, when the support weight map is used, the similarity measuring module 906 may use a radial cumulative similarity (RCS) function to remove a support weight existing outside the object in the symmetry window region.

The mid sagittal plane detecting module 908 detects a reference sagittal plane where the similarity between the symmetry window regions measured by the similarity measuring module 906 is highest, as the mid sagittal plane. In other words, the controller 113 may change the reference side image (or the reference sagittal plane) according to at least one of rotation transformation and translation transformation of the image data, and the similarity measuring module 906 may measure similarity according to changed reference side images. Accordingly, the mid sagittal plane detecting module 908 may analyze the result of repeatedly measured similarity, and detect the reference sagittal plane where the similarity is highest (i.e., where the symmetry window region is most similar), as the mid sagittal plane.

The determining of the mid sagittal plane is an important in analyzing the object through the image data or volume data. According to the configuration included in the controller 113 of the 3D ultrasound apparatus described above, the mid sagittal plane is semi-automatically detected, and thus the object may be easily and accurately diagnosed.

FIG. 10 is a flowchart illustrating a method for operating a 3D ultrasound apparatus according to another embodiment of the present invention. The method of FIG. 10 includes operations performed in time-series by the top image obtaining module 902, the symmetry window region detecting module 904, the similarity measuring module 906, and the mid sagittal plane detecting module 908 included in the controller 113 of FIG. 9. Accordingly, even if omitted in FIG. 10, details described with reference to FIG. 9 are applied to the method of FIG. 10.

In operation S1010, the 3D ultrasound apparatus determines the start point. The start point may be the virtual point which is spaced apart by a predetermined distance from a point at which a vertical line that passes through an end point of the fetus' nasal bone is intersected with a horizontal line that passes through the fetus' NT. Alternatively, the start point may be determined by a user input.

In operation S1030, the 3D ultrasound apparatus matches the template to the initial top image. In other words, the 3D ultrasound apparatus may determine the top image where the start point is located or the top image detected by the processor 111 as the initial top image (i.e., the initial transverse plane).

Then, the 3D ultrasound apparatus matches the template to the initial top image. For example, a parametric ellipse template may be used. A process of matching the template to the top image by the 3D ultrasound apparatus may include a) a denoising process as a pre-process, b) a thresholding process using a top-hat filter, c) a process of detecting an edge of an object (a fetus' head), d) a template matching process, and (3) a process of determining a parameter of the template.

According to the present embodiment, least squares fitting of a circle may be applied to the edge detected during the process of detecting the object, and a random sample consensus (RANSAC) may be further applied to remove an error. A modified chamfer matching technique may be applied as an example of the template matching process, and the parameter of the template may include a major axis radius and a minor axis radius of the template.

In operation S1050, the 3D ultrasound apparatus detects the symmetry window regions. As described above, the symmetry window regions are spaced apart by the same distance D from the reference sagittal plane, are parallel to each other, and have the length W. The distance D and the length W may be determined according to the parameter of the template obtained in operation S1030, and for example, may be represented according to Formulas 1 and 2 below.

$$\begin{matrix}W = \{ \begin{matrix}2 \cdot {Length}_{MajorAxis} & if {E}_{T} < {E}_{Th} \\ 2 \cdot {Length}_{Mean} & otherwise\end{matrix} & [ Formula 1 ] \\ D = \frac{1}{6} \cdot W & [ Formula 2 ]\end{matrix}$$

In Formulas 1 and 2, Length _{MajorAxis} denotes a major axis radius of the template and Length_{Mean} denotes an average radius of a fetus' head during a first trimester. E_{T} denotes template matching energy and E_{TH} denotes an experimentally determined value. IN other words, when the template matching energy is sufficiently low, the parameter of the template is reliable, but when the template matching energy is high, the 3D ultrasound apparatus may use the average experiment value.

The 3D ultrasound apparatus detects the two symmetry window regions parallel to the reference sagittal plane, and the reference sagittal plane may be a sagittal plane where the start point is located. The 3D ultrasound apparatus may change the reference sagittal plane by performing rotation transformation and translation transformation on the image data.

In operation S1070, the 3D ultrasound apparatus measures the similarity between the symmetry window regions. As described above, the example of the similarity function includes NCC function, and the 3D ultrasound apparatus may use any type of similarity function other than the NCC function. When the NCC function is used, a reference sagittal plane where an NCC value is highest is the mid sagittal plane.

Also, the 3D ultrasound apparatus may apply the support weight map about the pixels of the symmetry window regions along with the similarity function. The support weight map may be applied as a weight of the similarity function, and may be used to remove an outlier and noise. Also, the support weight map may include a probability function related to at least one of similarity consistency, distinctiveness, and proximity. The similarity consistency is a factor indicating a changed amount of similarity in the pixels in the symmetry window regions, and the distinctiveness is a factor about an anatomical boundary having a strong gradient, such as a nasal bone or a palate in the symmetry window regions. The proximity is a factor about a distance from the center of the symmetry window regions.

Also, if result values according to the similarity function and support weight map unnecessarily remain outside the object, the 3D ultrasound apparatus may use an RCS function to remove the result values. The RCS function may include an attribute similarity function using a diffusion operator and adaptive thresholding.

In operation S1090, the 3D ultrasound apparatus detects the mid sagittal plane. In other words, the 3D ultrasound apparatus may apply the similarity function while rotating and moving the image data based on a simulated annealing algorithm, and determine the reference sagittal plane where the similarity is highest as the mid sagittal plane.

FIGS. 11A and 11B are diagrams illustrating an example of detecting a mid sagittal plane according to the embodiment of the present invention. FIG. 11A illustrates the initial top image (the initial transverse plane), the reference sagittal plane, and the symmetry window regions, and FIG. 11B illustrates a relationship between the mid sagittal plane and the symmetry window regions.

In FIG. 11A, an image 1120 is the initial top image where a start point 1122 is located. Although not shown in FIG. 11A, the template may be matched to the top image as described above with reference to FIG. 7.

The 3D ultrasound apparatus may detect symmetry window regions 1126 and 1128 by using the parameter (length of size) of the template matched to the initial top image. The symmetry window regions 1126 and 1128 that are side images are displayed in lines in the top image, and are respectively shown in images 1110 and 1130.

The 3D ultrasound apparatus may determine similarity between the symmetry window regions 1126 and 1128 by using the similarity function and the support weight map, and determine a reference sagittal plane 1124 where the similarity is highest as the mid sagittal plane. In other words, the 3D ultrasound apparatus may measure the similarity while rotating or moving the image data, and detect the highest similarity.

In FIG. 11B, a start point 1145 is displayed in a reference sagittal plane 1140, and two symmetry window regions 1155 and 1165 are illustrated according to a length W and a distance D determined from the parameter of the template. The symmetry window regions 1155 and 1165 having the same lengths W are parallel to each other and are spaced apart from the reference sagittal plane 1140 by the same distance D. The symmetry window regions 1155 and 1165 may be respectively included in side images 1150 and 1160.

According to embodiments of the present invention, a top image of an object in a human body is extracted from an image data based on a start point in the image data obtained by scanning the object, and the image data is rotated using the extracted top image, thereby automatically determining a sagittal view of the object.

According to embodiments of the present invention, in a case where an object in a human body is a fetus, a top image of the object corresponding to a basic data on the rotation of an image data is easily extracted from the image data obtained by scanning the object, using the direction of the fetus' head, thereby rapidly controlling a sagittal view of the object.

The above-described exemplary embodiments of the present invention may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A method for operating a 3-dimensional (3D) ultrasound apparatus, the method comprising:
determining a start point from image data obtained by scanning an object in a human body; and
detecting a mid sagittal plane with respect to the object from the image data by using similarity between two symmetry window regions parallel to a reference side image where the start point is located.

2. The method of claim 1, further comprising changing the reference side image by rotating and moving the image data based on the start point.

3. The method of claim 1, wherein the detecting of the mid sagittal plane comprises detecting a reference side image where the similarity is highest as the mid sagittal plane.

4. The method of claim 1, further comprising:
matching a template with respect to the object indicated on an initial top image where the start point is located; and
obtaining the two symmetry window regions by using at least one of a length and a size of the template.

5. The method of claim 4, wherein the two symmetry window regions are located in the same distance from the reference side image, and the distance and the sizes of the two symmetry window regions are determined based on at least one of the length and the size of the template.

6. The method of claim 1, wherein the similarity is determined according to a similarity function based on a simulated annealing algorithm.

7. The method of claim 1, wherein the detecting of the mid sagittal plane comprises detecting the mid sagittal plane based on a support weight map and a similarity function with respect to pixels included in the two symmetry window regions.

8. The method of claim 7, wherein the support weight map comprises a probability function about at least one of similarity consistency, distinctiveness, and proximity of the two symmetry window regions.

9. The method of claim 8, wherein the detecting of the mid sagittal plane comprises applying a radial cumulative similarity function along with the similarity function and the support weight map.

10. A 3D ultrasound apparatus, the apparatus comprising:
a first processor to determine a start point from image data obtained by scanning an object in a human body; and
a controller to detect a mid sagittal plane with respect to the object from the image data by using similarity between two symmetry window regions parallel to a reference side image where the start point is located.

11. The apparatus of claim 10, wherein the controller changes the reference side image by rotating and moving the image data based on the start point.

12. The apparatus of claim 10, wherein the controller detects a reference side image where the similarity is highest as the mid sagittal plane.

13. The apparatus of claim 10, further comprising:
a second processor to match a template to an initial top image where the start point is located,
wherein the controller obtains the two symmetry window regions by using at least one of a length and a size of the template.

14. The apparatus of claim 13, wherein the two symmetry window regions are located in the same distance from the reference side image, and the distance and the sizes of the two symmetry window regions are determined based on at least one of the length and the size of the template.

15. The apparatus of claim 10, wherein the similarity is determined according to a similarity function based on a simulated annealing algorithm.

16. The apparatus of claim 10, wherein the controller detects the mid sagittal plane based on a support weight map and a similarity function with respect to pixels included in the two symmetry window regions.

17. The apparatus of claim 16, wherein the support weight map comprises a probability function about at least one of similarity consistency, distinctiveness, and proximity of the two symmetry window regions.

18. The apparatus of claim 17, wherein the controller applies a radial cumulative similarity function along with the similarity function and the support weight map.

19. A method for operating a 3D ultrasound apparatus, the method comprising:
determining a start point from image data obtained by scanning an object in a human body;
extracting a top image with respect to the object from the image data, based on the start point;
matching a template with respect to the object indicated on an initial top image where the start point is located; and
detecting a mid sagittal plane with respect to the object from the image data, by using similarity between symmetry window regions obtained based on the template and a reference side image where the start point is located.]

20. The method of claim 19, wherein the symmetry window regions are parallel to the reference side image, and are determined by using at least one of a size and a length of the template.

21. The method of claim 19, further comprising changing the reference side image by rotating and moving the image data based on the start point,
wherein the mid sagittal plane is a reference side image where the similarity is highest, which is a result of a similarity function based on a simulated annealing algorithm.

22. The method of claim 19, wherein the detecting of the mid sagittal plane comprises detecting the mid sagittal plane based on a support weight map and a similarity function, wherein the support weight map is a function about at least one of similarity consistency, distinctiveness, and proximity of the symmetry window regions.

23. The method of claim 22, wherein the detecting of the mid sagittal plane comprises applying a radial cumulative similarity function, along with the similarity function and the support weight map.

24. A 3D ultrasound apparatus, the apparatus comprising:
a first processor to determine a start point from image data obtained by scanning an object in a human body;
a second processor to extract a top image with respect to the object from the image data, based on the start point, and match a template with respect to the object indicated on an initial top image where the start point is located; and
a controller to detect a mid sagittal plane with respect to the object from the image data, by using similarity between symmetry window regions obtained based on the template and a reference side image where the start point is located.

25. The apparatus of claim 24, wherein the symmetry window regions are parallel to the reference side image, and are determined by using at least one of a size and a length of the template.

26. The apparatus of claim 24, wherein the controller changes the reference side image by rotating and moving the image data based on the start point,
wherein the mid sagittal plane is a reference side image where the similarity is highest, which is a result of a similarity function based on a simulated annealing algorithm.

27. The apparatus of claim 24, wherein the controller detects the mid sagittal plane based on a support weight map and a similarity function, wherein the support weight map is a function about at least one of similarity consistency, distinctiveness, and proximity of the symmetry window regions.

28. The apparatus of claim 27, wherein the controller applies a radial cumulative similarity function, along with the similarity function and the support weight map.
